(19) United States
(12) Patent Application Publication
(10) Pub. No.: US 2009/0234027 A1
(43) Pub. Date: Sep. 17, 2009

(54) Topical gel cosmetic formulation of cellacefate for the control of acne and rosacea

(76) Inventor: Nathan Strick, Oceanside, NY (US)

Correspondence Address: Thomas A. O'Rourke;Bodner & O'Rourke, 425 Broadhollow Road, Melville, NY, 11747, US

(21) Appl. No.: 12/378,289

(22) Filed: Feb. 12, 2009

Related U.S. Application Data
(60) Provisional application No. 61/069,653, filed on Mar. 14, 2008.

(51) Int. Cl. A61K 47/38; C07H 3/00; A61P 17/10

(52) U.S. Cl. 514/781; 536/63

(57) ABSTRACT

Compositions for the treatment of acne or rosacea on the skin are described. Also provided are methods of forming the compositions as well as the treatment itself. The composition includes as an active ingredient cellulose acetate phthalate (CAP).

This application claims priority on U.S. Provisional Patent Application Ser. No. 61/069,653 filed Mar. 14, 2008, the disclosures of which are incorporated herein by reference.

### FIELD OF THE INVENTION

The present invention relates to the field of cosmetic formulations useful in treating skin conditions such as acne, rosacea and the like.

### BACKGROUND OF THE INVENTION

Acne is an inflammatory condition of the skin usually starting at puberty and lasting many years. It may appear on the face, chest or back or in any combination of the three. It varies in severity from mild to disfiguring. At any level of severity it can cause great psychological damage and if scarring occurs, the loss of self-esteem can be devastating. Mild cases of acne may respond to salicylic acid or benzoyl peroxide, both sold over the counter. More severe cases are treated with prescription medications such as antibiotics, topicals and as a last resort, isotretinoin. Available over the counter products are not very effective and are sometimes irritants. Prescription medications are not always effective, and can have dangerous side effects. Antibiotics can lead to drug resistant bacteria.

Rosacea is a chronic inflammatory skin condition usually starting in middle age and resembling acne. It mostly appears on the central area of the face and may cause tissue hypertrophy, especially on the nose. Since people with rosacea tend to look like alcoholics, there are esthetic, social and psychological costs to the sufferer. The treatment for rosacea is similar to that for acne.

While the foregoing treatments provide results in some applications, there is a need for a treatment that provides more consistent results in a wide variety of patient conditions. Thus, there is a need for finding a composition that will control skin conditions such as acne and rosacea but which also has microbiocidal and anti-inflammatory activity, a history of safe use, and that can be applied topically to a patient.

### SUMMARY OF THE INVENTION

The present invention is directed to a composition for the treatment of various skin conditions. The skin conditions include acne, rosacea and the like. The composition of the present invention is a topical gel that includes cellulose acetate phthalate (CAP) as the primary ingredient. The gel is applied to the skin and permitted to remain in contact with the skin. In a preferred embodiment, the composition is applied to the skin and remains in contact with the skin for up to eight hours or so. The longer the composition of the present invention is in contact with the skin the better the results.

### BRIEF DESCRIPTION OF THE DRAWING

FIG. 1 shows a chemical formula for cellulose acetate phthalate (CAP).

### DETAILED DESCRIPTION OF THE INVENTION

In accordance with the present invention there has been found that improved results in treating skin conditions such as acne and rosacea can be achieved by compositions containing a blend based on Cellacefate, also called, cellulose acetate phthalate (CAP). CAP has a long history of safe use as an enteric coating. Cellulose acetate phthalate is used as an enteric coating on capsules or tablets so they don't dissolve until they reach the small intestine. Enteric coatings are typically, selectively insoluble substances, i.e., they don't dissolve in the acidic juices of the stomach, but they will dissolve in the higher pH (above pH 5.5) of the small intestine. CAP is not irritating, not a teratogen nor a carcinogen or mutagen. Large doses have been fed to animals with no harmful effects. These results have been documented in the scientific literature.

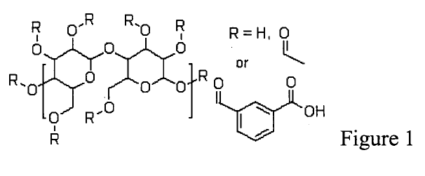

CAP is a cellulose polymer with substituted phthalates at the hydroxyl positions. The compound has the structure shown in FIG. 1.

CAP is a mixed ester of cellulose, acetic acid and phthalic acid. CAP is a reaction product of phthalic anhydride and a partial acetate ester of cellulose; used as a tablet-coating agent. The molecular weight is 50-60 kD. A water suspension gives a pH between 2 and 3. It is insoluble in water at a pH below 5 but becomes somewhat soluble at higher pH. At neutral and higher pH it becomes unstable. Strong bases are not suitable to raise the pH. Sodium acetate at 0.33 M will dissolve CAP to 3% but higher amounts salt out. The solutions are also not very stable. The best system is to dissolve the CAP using a readily available and cheap chemical, sodium bicarbonate. Either powder or a concentrated solution of sodium bicarbonate or both can be added to a suspension of CAP.

CAP is available commercially in several forms, as a granular solid (Cellacefate), a micronized solid (Aquateric), and as a micronized water suspension (Aquacoat). Aquateric is about 70% CAP, the balance being poloxamer 188 and acetylated monoglycerides. Aquacoat contains about 30% solids, 19-27% CAP and 3-11% poloxamer 188, the balance being water. Granular CAP is too coarse to formulate and must be dissolved in some way. Aquateric and Aquacoat are relatively easy to formulate so long as the system is compatible with all their components.

A preferred formulation of the present invention can be made by suspending CAP in deionized or distilled water. The preferred composition has 6 wt % CAP to about 7 wt % CAP in water. The composition is formed by adding CAP to the water. The pH of the blend must be adjusted to cause the CAP to dissolve in the water. The pH is adjusted by slowly adding sodium bicarbonate into the solution with constant stirring. The stirring allows carbon dioxide to evolve from the suspension. The amount of sodium bicarbonate added to CAP suspensions is dependent on the starting material, and because of batch differences in acidity, needs to be determined empirically. The sodium bicarbonate is preferably between about 0.5 to about 1.5 weight % of the final solution. Sodium bicarbonate is added until all the CAP dissolves.

For reasons of efficiency Aquacoat and Aquateric are more convenient as the starting ingredient but granulated CAP allows more flexibility in formulation. The suspension contains about 5-8 weight % CAP final composition, more preferably about 6-7%. The CAP solution may contain 0-3 weight % poloxamer 188 and 0-1 weight % acetylated monoglycerides but 0% is preferred for both. A bioadhesive such as methylcellulose (MC) or hydroxypropylmethylcellulose (HPMC) of viscosity 4000 cPs at 2-3 weight %, preferably HPMC at 2.5 weight %, is slowly added with mixing to the composition so that a viscous gel forms. A preservative such as salicylic acid may also be used. The salicylic acid is preferably present in a range of 0 to about 2.0% and more preferably present in a range of about 0.4% to about 0.6% by weight. Other preservatives can include potassium sorbate, sodium benzoate or their acid forms, 0.1 weight % each, should be used in the composition. Methyl paraben and propyl paraben to 0.13 weight % combined total may be added as well. The added ingredients are common excipients. The clear gel now formed can be easily spread over the skin and adheres tightly. Aquateric is the least suited for this procedure in that the acetylated monoglycerides are oily and not water soluble, instead forming a milky suspension.

The described gel works very well in controlling acne and rosacea. By the term acne and rosacea is meant acne vulgaris and acne rosacea. The composition is also effective in treating seborrheic dermatitis. In tests (20 of 20 and 2 of 2 people respectively) if properly used, the gel worked well in controlling acne and rosacea. The CAP gel should be applied to the area being treated daily and left on for about 8 hours. Before going to sleep is a convenient time. It can then be washed off. Preferably the treatment is performed every day for the first week. After the first week, the treatment is preferably applied every other day. The treatment should continue as long as the symptoms continue. Even after the symptoms have been eliminated, treatment should continue every second or third day to ensure that the symptoms do not reoccur. In summary, a novel formulation is described whose active ingredient is a macromolecule, CAP, which is known to be safe from years of testing and use. The novel chemical form of the ingredient as used, a soluble salt, allows its formulation as a clear gel. This gel can make intimate contact with the skin and strongly adheres, but it does not penetrate into cells. It is not a cure for acne or rosacea but has controlled the symptoms of both very successfully. All the ingredients are generally recognized as safe.

I claim:

1. A skin treatment composition for the treatment of skin disorders comprising a gel comprising cellulose acetate phthalate.

2. The composition according to claim 1 wherein said cellulose acetate phthalate comprises about 5 to about 8 weight percent of the composition.

3. The composition according to claim 2 wherein said composition further comprises a bioadhesive.

4. The composition according to claim 3 wherein said bioadhesive is methylcellulose.

5. The composition according to claim 3 wherein said bioadhesive is hydroxypropyl methylcellulose.

6. The composition according to claim 3 where said bioadhesive comprises about 2-3 weight percent of the composition.

7. The composition according to claim 6 further comprising a preservative.

8. The composition according to claim 7 wherein said preservative is salicylic acid

9. A method of forming a gel for the treatment of skin disorders comprising blending cellulose acetate phthalate (CAP) in water and adjusting the pH of the blend to a pH of 5 or higher.

10. The method according to claim 9 wherein said pH is adjusted using sodium bicarbonate.

11. The method according to claim 10 wherein sodium bicarbonate is added into the blend of cellulose acetate phthalate (CAP) accompanied by constant stirring of the solution.

12. The method according to claim 10 wherein said sodium bicarbonate is a concentrated solution of sodium bicarbonate and water.

13. The method according to claim 10 wherein said sodium bicarbonate is a powder.

14. The method according to claim 11 further comprising a bioadhesive.

15. The method according to claim 14 wherein said bioadhesive is methylcellulose.

16. The method according to claim 14 wherein said bioadhesive is hydroxypropyl methylcellulose

17. The method according to claim 9 wherein said cellulose acetate phthalate (CAP) is a granular solid.

18. The method according to claim 9 wherein said cellulose acetate phthalate (CAP) is a micronized solid.

19. The method according to claim 9 wherein said cellulose acetate phthalate (CAP) is a micronized water suspension.

20. A method of treating acne or rosacea on the skin comprising administering a therapeutically effective amount of cellulose acetate phthalate (CAP) in a vehicle suitable for topical application to the skin to a patient with acne or rosacea.

21. The method according to claim 20 wherein said vehicle is a gel.

22. The method according to claim 21 wherein said gel containing said cellulose acetate phthalate has a pH of 5 or higher.
